(19) United States
(12) Patent Application Publication
(10) Pub. No.: US 2007/0054841 A1
(43) Pub. Date: Mar. 8, 2007

(54) Method of treatment of systemic injury secondary to burns

(75) Inventors: Ian Alexander Shiels, Queensland (AU); Stephen Maxwell Taylor, Queensland (AU); Shelli Z. Stocks, Queensland (AU)

Correspondence Address: HAYNES AND BOONE, LLP, 901 MAIN STREET, SUITE 3100, DALLAS, TX, 75202, US

(73) Assignee: The University of Queensland, Brisbane (AU)

(21) Appl. No.: 10/557,498

(22) PCT Filed: May 26, 2004
(86) PCT No.: PCT/AU04/00703

§ 371 Date: Nov. 14, 2006

(30) Foreign Application Priority Data
May 26, 2003 (AU) 2003902586

(51) Int. Cl. A61K 38/12

(52) U.S. Cl. 514/9

(57) ABSTRACT

The invention relates to the prevention or treatment of a systemic injury which is secondary to a burn, such as dysfunction or failure of an organ secondary to a burn, with an antagonist of a C5a receptor. In one embodiment the invention relates to the prevention or treatment of dysfunction or failure of the lung, kidney, bowel and/or liver which is secondary to a burn.

### RELATED APPLICATIONS

This application claims priority from Australian provisional patent application 2003902586 which was filed on 26 May 2003; the entire contents of which are hereby incorporated by cross-reference.

### FIELD OF THE INVENTION

This invention relates to use of an antagonist of a C5a receptor for the prevention or treatment of a systemic injury which is secondary to a burn, such as dysfunction or failure of an organ secondary to a burn. In one embodiment the invention relates to the prevention or treatment of dysfunction or failure of the lung, kidney, bowel and/or liver which is secondary to a burn.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Systemic injury, such as the dysfunction or failure of an organ secondary to a severe burn injury and which is not attributable to the burn injury, remains a continuing source of morbidity and mortality, and is of particular relevance in the military environment. There is currently no recognised effective treatment or means of prevention for such organ failure, other than supportive care to compensate for the decreased organ function. In particular, dysfunction or failure of the lung following burns to the skin or other sites of the body has a significant impact on morbidity and mortality. Dysfunction of the liver, kidneys and/or bowel is also a possible outcome of burns and this also leads to a poorer prognosis in morbidity and mortality.

It is thought that systemic inflammatory responses arise in subjects following burn injury, and that it is this generalised inflammation which leads to the remote tissue injury which is expressed as the dysfunction and failure of organs remote from the injury site.

The chain of physiological processes which lead to organ failure following burns is complex, and neither fully understood nor fully characterized. In subjects with serious burns, release of catecholamines, vasopressin, and angiotensin causes peripheral and splanchnic bed vasoconstriction that can compromise perfusion of organs remote to the injury. Myocardial contractility also may be reduced by the release of TNF-α. Activated neutrophils are sequestered in dermal and distant organs such as the lung within hours following a burn injury, resulting in the release of toxic reactive oxygen species and proteases and producing vascular endothelial cell damage. When the integrity of pulmonary capillary and alveolar epithelia is compromised, plasma and blood leak into the interstitial and intra-alveolar spaces, resulting in pulmonary oedema. A decrease in pulmonary function can occur in severely burned patients, as a result of bronchoconstriction caused by humoral factors, such as histamine, serotonin, and thromboxane A2.

Severe burn injury also causes a coagulation necrosis of tissue. This initiates a physiological response in every organ system, the severity of which is related to the extent of the burn. Tissue destruction also results in increased capillary permeability, with profound egress of fluid from the intravascular space to the tissues adjacent to the burn wound. Inordinate amounts of fluid are lost by evaporation from the damaged surface, which is no longer able to retain water. This increase in capillary permeability, coupled with evaporative water loss, causes a hypovolaemic shock, which may also in turn contribute to remote organ dysfunction or failure.

Compounds which have been implicated in the pathogenesis of remote organ dysfunction or failure include a broad range of humoral mediators, such as various components of complement; products of arachidonic acid metabolism, such as products of lipoxygenase or cyclooxygenase enzymes; tumor necrosis factor; cytokines, such as interleukins 1 to 13; a range of growth factors and adhesion molecules; platelet activating factor; procoagulants; fibronectin and opsonins; toxic oxygen free-radicals; endogenous opioids such as endorphins; vasoactive polypeptides and amines; bradykinin and other kinins; neuroendocrine factors; myocardial depressant factor and coagulation factors and their degradation products. In addition, various cellular components of inflammation have been implicated in the systemic responses which lead to organ dysfunction, including polymorphonuclear leukocytes (PMNLs) which are transiently sequestered in the blood vessels of the organs of the burned subject; monocytes and macrophages which may release a variety of inflammatory mediators; platelets; and vascular endothelial cells, which mediate the passage of fluids and solutes between the vasculature and the organs.

The role of nitric oxide in burn-related pulmonary dysfunction has also been investigated. Increased production of nitric oxide has been observed in human burn patients and in animal models of thermal injury. Inducible nitric oxide synthase (iNOS) may mediate pulmonary inflammation and tissue injury following large cutaneous burns. It has been speculated that inhibition of nitric oxide synthase activity could be of benefit in the therapy of the acute post-burn inflammatory response.

It has also been reported that the administration of neutralizing antibodies to C5a significantly blocked lung injury following experimental burns in rats (Schmid E, Piccolo M T, Friedl H P, Warner R L, Mulligan M S, Hugli T E, Till G O, Ward P A. (1997) Requirement for C5a in lung vascular injury following thermal trauma to rat skin. Shock 8:119-124).

To date, there is currently no accepted effective drug for therapeutic or preventative use in the prevention or treatment of organ dysfunction or failure following burns. Burns patients receive a regimen of supportive care which involves pain management, fluid replacement and care aimed at prevention of gastric erosion and prevention of renal failure. Acute upper gastrointestinal tract erosions and ulcers may occur in patients with severe burn injuries, and treatment is principally preventive. In high-risk patients, antacids can reduce the occurrence of stress ulcerations by neutralizing gastric contents, and H₂-receptor antagonists can inhibit gastric acid secretion. Renal failure can occur after burn injury, and its prevention involves adequate resuscitation, treatment of infection in the wound and other sites, and avoidance of nephrotoxic drugs. When renal function deteriorates with resultant fluid and electrolyte imbalance, dialysis may be required. Subjects with lung dysfunction or failure may require artificial ventilation in order to maintain sufficient oxygen delivery to the body. Symptoms of organ dysfunction or failure may become apparent shortly after burning or within 6 to 18 hours following the burn, and may progress until supportive therapy is instituted.

Accordingly, there is still a great need for a method of therapeutic or preventative intervention for systemic injury, such as organ dysfunction or failure which arises from burn distant to the organ, to supplement or replace the supportive interventions currently employed.

### SUMMARY OF THE INVENTION

We have now found that a compound, PMX53, from a family of C5a receptor antagonists, has a disease-modifying effect on lung dysfunction or failure following burn injury.

According to a first aspect, the invention provides a method of treatment of a systemic injury secondary to burns, comprising the step of administering to a subject in need thereof a therapeutically or prophylactically effective amount of a compound which is an antagonist of a C5a receptor and which is a cyclic peptide or peptidomimetic compound of Formula I

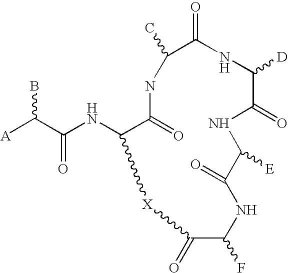

where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;

B is an alkyl, aryl, phenyl, benzyl, naphthyl or indole group, or the side chain of a D- or L-amino acid such as L-phenylalanine or L-phenylglycine, but is not the side chain of glycine, D-phenylalanine, L-homophenylalanine, L-tryptophan, L-homotryptophan, L-tyrosine, or L-homotyrosine;

C is a small substituent, such as the side chain of a D-, L- or homo-amino acid such as glycine, alanine, leucine, valine, proline, hydroxyproline, or thioproline, but is preferably not a bulky substituent such as isoleucine, phenylalanine, or cyclohexylalanine;

D is the side chain of a neutral D-amino acid such as D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine, but is preferably not a small substituent such as the side chain of glycine or D-alanine, a bulky planar side chain such as D-tryptophan, or a bulky charged side chain such as D-arginine or D-Lysine;

E is a bulky substituent, such as the side chain of an amino acid selected from the group consisting of L-phenylalanine, L-tryptophan and L-homotryptophan, or is L-1-napthyl or L-3-benzothienyl alanine, but is not the side chain of D-tryptophan, L-N-methyltryptophan, L-homophenylalanine, L-2-naphthyl L-tetrahydroisoquinoline, L-cyclohexylalanine, D-leucine, L-fluorenylalanine, or L-histidine;

F is the side chain of L-arginine, L-homoarginine, L-citrulline, or L-canavanine, or a bioisostere thereof, ie. a side chain in which the terminal guanidine or urea group is retained, but the carbon backbone is replaced by a group which has different structure but is such that the side chain as a whole reacts with the target protein in the same way as the parent group; and

X is —(CH₂)ₙNH— or (CH₂)ₙ—S—, where n is an integer of from 1 to 4, preferably 2 or 3; —(CH₂)₂O —; —(CH₂)₃O—; —(CH₂)₃—; —(CH₂)₄—; —CH₂COCHRNH—; or —CH₂—CHCOCHRNH—, where R is the side chain of any common or uncommon amino acid.

In C, both the cis and trans forms of hydroxyproline and thioproline may be used.

Preferably A is an acetamide group, an aminomethyl group, or a substituted or unsubstituted sulphonamide group.

Preferably where A is a substituted sulphonamide, the substituent is an alkyl chain of 1 to 6, preferably 1 to 4 carbon atoms, or a phenyl or toluyl group.

In a preferred embodiment, the systemic injury is organ dysfunction or failure.

In one embodiment the application provides a method for the treatment of a systemic injury secondary to burns, comprising the step of administering a therapeutically or prophylactically effective amount of compound 1 (PMX53; AcF-[OPdChaWR]), compound 33 (AcF[OP-DPhe-WR]), compound 60 (AcF[OP-DCha-FR]) or compound 45 (AcF[OP-DCha-WCit]) described in International Patent Application No. PCT/AU02/01427, or HC-[OPdChaWR] (PMX205), AcF-[OPdPheWR] (PMX273), AcF-[OPdChaWCitrulline] (PMX201) or HC-[OPdPheWR] (PMX218).

In one aspect the compound is an antagonist of C5a receptors on human and/or mammalian cells including, but not limited to, human polymorphonuclear leukocytes and/or human macrophages. In certain embodiments, the compound is an antagonist of Class I C5a receptors.

In some aspects the compound binds potently and selectively to C5a receptors, and for instance has potent antagonist activity at sub-micromolar concentrations. Even more preferably the compound has a C5a receptor affinity IC₅₀ of less than or equal to 25 μM, and an antagonist potency IC₅₀of less than 1 μM.

In some embodiments, the compound has an antagonist activity against a C5a receptor, and has no detectable C5a agonist activity.

In another aspect, the present application provides a use of a compound as described above for treating and or preventing organ dysfunction or failure arising from burns.

In another aspect there is provided a use of a compound as described above for the preparation of a medicament for use in preventing or treating systemic injury, such as organ dysfunction or failure secondary to burns.

In another aspect there is provided a pharmaceutical or veterinary agent for preventing or treating systemic injury, such as organ dysfunction or failure secondary to burns, comprising a compound as described above.

In another aspect there is provided a composition for preventing or treating systemic injury, such as organ dysfunction or failure secondary to burns, comprising a compound as described above together with a pharmaceutically or veterinarily-acceptable carrier.

In some embodiments, the organ is lung, liver, kidney and/or bowel.

### BRIEF DESCRIPTION OF THE FIGURES

In the examples hereinafter, reference will be made to the accompanying figures as follows:

FIG. 1 is a photograph which illustrates the leakage of Evans Blue into rat skin 4 hours after a thermal skin burn. The two samples on the left are from rats which received a burn only, with no drug treatment, the middle sample is from a no burn, no treatment control, and the two samples on the right are from rats which were pretreated with PMX53 (10 mg/kg SC 30 minutes) prior to burning. PMX53-pretreated rats showed markedly less plasma leakage into the skin, as indicated by the lesser intensity of the dark colour.

FIG. 2 is a photograph which demonstrates leakage of Evans Blue/albumin into the subcutaneous tissues of burned rats. The two skin samples on the top are from burn-only rats. The two skin samples below them are from rats pretreated with PMX53, and the skin sample on the right is from a normal control rat.

FIG. 3 is a photograph which shows the macroscopic appearance of the lung 4 hours after burn. The two lungs on the left were from burn-only control rats, the middle lung was a no-burn control, and the two lungs on the right were from burned rats pretreated with PMX53 10 mg/kg SC. The burn-only lungs show extensive consolidation, whilst lungs from PMX53-pretreated rats appear normal.

FIG. 4 is a graph illustrating the total number of cells recovered from bronchoalveolar lavage (BAL) from rats at 4 hours after a burn injury. Whilst the BAL fluid from untreated lungs of burned animals contained relatively high numbers of cells, the burned animal treated with PMX53 showed cell numbers approaching those found in the sham-operated, unburned animals.

FIG. 5 is a graph which shows the effect of pretreatment with PMX53 10 mg/kg SC 30 minutes prior to the burn on lung myeloperoxidase (MPO) levels at 4 hours after burning. The elevated levels of MPO indicate the presence of neutrophils in the lung.

FIG. 6 shows photomicrographs of rat skin samples following burns to illustrate tissue damage and the distribution of PMNLs 6 hours after a burn. Photomicrograph A is of a sample of normal, unburned skin, B is of burned and untreated skin and C is of burned skin which was treated with topically administered PMX53.

FIG. 7 is a graph which illustrates the effect of topical administration of PMX53 on the level of IgG in plasma 6 hours after a burn.

### DETAILED DESCRIPTION OF THE INVENTION

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

In the present specification, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It must be noted that, as used in the present specification, the singular forms “a”, “an” and “the” include plural aspects unless the context clearly dictates otherwise. Thus, for example, reference to the use of “a compound” includes the use of a single compound, as well as the use of two or more compounds; and so forth.

It is contemplated that the compounds described in this application may be administered to a subject following a burn but before the development of detectable symptoms of a systemic injury such as organ dysfunction or failure, and thus the term “prevention” is used herein in its broadest sense and refers to a prophylactic use which completely or partially prevents systemic injury, such as organ dysfunction or failure or a sign or symptom thereof following burns. It is contemplated that the compounds may be administered to a subject at risk of receiving burns.

It is also contemplated that the compounds described in this application may be administered to a subject following a burn and after the onset of detectable symptoms of systemic injury, or that administration may continue from previous prophylactic administration of the compound. Thus the term “treatment” is used herein in its broadest sense and refers to use of a compound for a partial or complete cure of organ dysfunction or failure. “Treating” as used herein covers any treatment of, or prevention of a condition in a vertebrate, a mammal, particularly a human, and includes inhibiting the condition, i.e., arresting its development; or relieving or ameliorating the effects of the condition, i.e., causing regression of the effects of the condition.

The term “organ” as used herein refers to a part or structure of the body which is adapted for a special function or functions, and includes but is not limited to the lungs, the liver, the kidneys, and the bowel, including the stomach and intestines. In particular, it is contemplated that organs which are particularly susceptible to dysfunction and failure arising from a burn to another part of the body are encompassed by this term.

“Organ dysfunction” as used herein refers to a continuum of indications ranging from a minor perturbation in the normal function(s) of an organ to “organ failure” ie. the cessation of sufficient organ output to sustain life. In the lung, for example, organ dysfunction may present as a decrease in pulmonary function caused by diminishing of pulmonary and tissue compliance. In the early post-resuscitation period, usually two to six days following burn injury, major abnormalities that impair pulmonary function include pulmonary oedema, upper airway obstruction and decreased chest wall compliance. In the heart, the response to thermal injury is a reduction in cardiac output which is accompanied by an increase in peripheral vascular resistance. Cardiac failure can result from a direct effect on myocardial contractility from the release of the inflammatory cytokine, TNF, and an indirect action of the tissue hypoxia resulting from the reduction in oxygen perfusion in peripheral tissues. Vascular complications, such as thrombophlebitis, can cause secondary ischaemic disorders in the extremities. In the kidney, organ dysfunction may manifest as an inability to excrete ion loads, leading to systemic ion imbalances. In the central nervous system, hyponatraemia may result from the rehydration therapy, which may lead to cerebral oedema and possibly post-burn encephalopathy. In the digestive tract several forms of complications may occur following severe burns. A haemorrhagic syndrome is caused by the action of gastric acids on the stomach mucosa, and can develop into a condition called Curling's ulcer. Another possible complication is paralytic ileus, which is caused by a decrease in intestinal motility and integrity.

In many organs, organ dysfunction may result from decreased organ blood flow, an increased burden of PMNLs located in the organ vasculature and surrounding tissue, and an increased vascular permeability.

Subjects suffering from severe burns are also at great risk of sepsis. Bacterial invasion occurs in a burn patient because the skin no longer acts as a barrier to the entrance of microorganisms. Because of their reduced ability to mount an effective systemic immune response, severely burned patients are susceptible to the development of sepsis and life-threatening septic shock. Sepsis is, however, a separate complication from the organ dysfunction or failure which occurs secondary to burns. Organ dysfunction or failure secondary to burns may occur in the absence of sepsis.

A characteristic of the systemic injury, organ dysfunction or organ failure contemplated by the present invention is that the burn which provokes the subsequent injury, dysfunction or failure does not directly affect the organ in question, ie. the injury is secondary to the burn. Without wishing to be bound by any theoretical mechanism, it is proposed that a systemic inflammatory response which arises as a result of the burn is the underlying cause of the subsequent dysfunction or organ failure.

It is contemplated that the invention is applicable to the treatment of systemic injury, such as organ dysfunction or failure arising from burns from any cause, including dry heat or cold burns, scalds, sunburn, electrical burns, chemical agents such as acids and alkalis, including hydrofluoric acid, formic acid, anhydrous ammonia, cement, and phenol, or radiation burns. Burns resulting from exposure to either high or low temperature are within the scope of the invention. The severity and extent of the burn may vary, but secondary organ dysfunction or failure will usually arise when the burns are very extensive or very severe (second or third degree burns). The development of secondary organ dysfunction or failure is dependent on the extent of the burn, the response of the patient's immune system and other factors such as infection and sepsis.

The term “antagonist” as used herein refers to the ability of the described compounds to inhibit C5a activity. Without wishing to be bound by any proposed mechanism, it is thought that the C5a receptor antagonists described in the present application are competitive inhibitors of C5a that act by binding to the C5a receptor. The antagonist activity of these compounds may be quantified by using a receptor binding assay, such as that described in the general methods section of this specification. Antagonist potency is indicated by activity at a concentration in the nanomolar range. Specificity is indicated by the inactivity of the compound at low concentration on other types of receptors. The preferred compounds of the invention have a high level of selectivity, with an IC₅₀ greater than 100 μM against formylated met-leu-phe, leukotriene B₄— or platelet activating factor-induced enzyme release.

Conversely, the phrase “substantially no agonist activity” as used herein refers to the inability of the compounds to induce signal transduction events from the C5a receptor which lead to physiological outcomes associated with this receptor's activation, such as activation of PMNLs, an increase in vascular permeability and the production of a variety of inflammatory mediators. The compound PMX53 is devoid of detectable agonist activity, as monitored in sensitive assays for chemotaxis and polarisation of neutrophils. (Finch AM et al: Low molecular weight peptidic and cyclic antagonists of the receptor for the complement factor C5a. J Med Chem 42: 1965-1974, 1999). A quantitative measure of this activity may be made using the myeloperoxidase release assay which is described in the general methods section of this application.

Throughout the specification conventional single-letter and three-letter codes are used to represent amino acids.

Other abbreviations used herein are as follows:
BAL bronchoalveolar lavage
Cit citrulline
dCha D-cyclohexylamine
DPhe D-phenylalanine
EB Evans Blue
lg immunoglobulin
IL-6 interleukin-6
ip intraperitoneal
iv intravenous
LPS lipopolysaccharide
MAP mean arterial pressure
MPO myeloperoxidase
PMNL polymorphonuclear leucocytes (polymorphonuclear granulocytes)
PMSF phenylmethylsulfonyl fluoride
sc subcutaneous
TNF-α tumour necrosis factor-α

A “common” amino acid is an L-amino acid selected from the group consisting of glycine, leucine, isoleucine, valine, alanine, phenylalanine, tyrosine, tryptophan, aspartate, asparagine, glutamate, glutamine, cysteine, methionine, arginine, lysine, proline, serine, threonine and histidine.

An “uncommon” amino acid includes, but is not restricted to, D-amino acids, homo-amino acids, N-alkyl amino acids, dehydroamino acids, aromatic amino acids other than phenylalanine, tyrosine and tryptophan, ortho-, meta- or para-aminobenzoic acid, ornithine, citrulline, canavanine, norleucine, γ-glutamic acid, aminobutyric acid, L-fluorenylalanine, L-3-benzothienylalanine, and α,α-disubstituted amino acids.

For the purposes of this specification, the term “alkyl” is to be taken to mean a straight, branched, or cyclic, substituted or unsubstituted alkyl chain of 1 to 6, preferably 1 to 4 carbons. Most preferably the alkyl group is a methyl group.

The term “acyl” is to be taken to mean a substituted or unsubstituted acyl of 1 to 6, preferably 1 to 4 carbon atoms. Most preferably the acyl group is acetyl.

The term “aryl” is to be understood to mean a substituted or unsubstituted homocyclic or heterocyclic aryl group, in which the ring preferably has 5 or 6 members.

The compounds described in this application may be used in conjunction with one or more other agents useful for the treatment of burns, including but not limited to general supportive measures such as intravenous fluids and administration of analgesic drugs and antibiotics.

The compositions described in this application may be formulated for oral, parenteral, inhalational, intranasal, rectal, or transdermal use, but oral or topical formulations are preferred. It is expected that most if not all of the compounds will be stable in the presence of metabolic enzymes, such as those of the gut, blood, lung or intracellular enzymes. Such stability can readily be tested by routine methods known to those skilled in the art.

The compounds described in this application may be administered at any suitable dose and by any suitable route. Oral or transdermal administration is preferred, because of the greater convenience and acceptability of these routes. The effective dose will depend on the nature of the condition to be treated, and the age, weight, and underlying state of health of the individual treatment. This will be at the discretion of the attending physician or veterinarian. Suitable dosage levels may readily be determined by trial and error experimentation, using methods which are well known in the art.

Representative compounds described in this application, including PMX53, have been demonstrated to remain stable and active following incubation in serum at 37° C. for 1 hour.

It is contemplated that dosages of the compound for humans will be in the ranges of from 0.5 to 20 mg/kg body weight for oral application, preferably from 1.0 to 10 mg/kg body weight, from 0.1 to 1 mg/kg body weight for intravenous administration, from 0.1 to 10 mg/kg for subcutaneous administration, and 10 mg/ml gel for topical administration routes.

Previous studies have demonstrated that oral administration of PMX53 at 100 mg/kg body weight/day to rats did not attenuate or increase T-cell dependent antigen responses in a sheep red blood cell ISHQ assay. Oral administration of PMX53 at 100 mg/kg body weight/day also did not detectably impair the phagocytosis or the oxidative burst functions of granulocytes in cynomolgus monkeys.

Suitable formulations for administration by any desired route may be prepared by standard methods, for example by reference to well-known textbooks such as Remington: The Science and Practice of Pharmacy, Vol. II, 2000 (20^{th} edition), A. R. Gennaro (ed), Williams & Wilkins, Pennsylvania.

While the methods according to the invention are not in any way restricted to the treatment of any particular animal or species, it is particularly contemplated that the methods will be useful in medical treatment of humans, and will also be useful in veterinary treatment, particularly of companion animals such as cats and dogs, livestock such as cattle, horses and sheep, and zoo animals, including non-human primates, large bovids, felids, ungulates and canids.

The use of various pharmaceutical compositions for ameliorating disease is described in certain embodiments. The pharmaceutical compositions according to one embodiment are prepared by bringing a compound of formula I, analogue, derivatives or salts thereof and one or more pharmaceutically-active agents or combinations of compound of formula I and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to certain embodiments may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg. in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) a naturally occurring phosphatide such as lecithin;

(b) a condensation product of an alkylene oxide with a fatty acid, for example, polyoxyethylene stearate;

(c) a condensation product of ethylene oxide with a long chain aliphatic alcohol, for example, heptadecaethylenoxycetanol;

(d) a condensation product of ethylene oxide with a partial ester derived from a fatty acid and hexitol such as polyoxyethylene sorbitol monooleate, or

(e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono-or diglycerides. In addition, fatty acids such as oleic acid may be used in the preparation of injectables.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

It may be advantageous to administer the compounds described in this specification topically via a bandage or dressing which is applied to the burn injury. In such cases, the compounds may be formulated in a gel or encapsulated form using standard techniques well known in the art. Topically-administered PMX53 at concentrations of 10 mg/ml gel was found to be well tolerated and safe over 56 days in subjects participating in an unrelated clinical trial.

Dosage levels of the compound of formula I of the present invention will usually be of the order of about 0.5 mg to about 20 mg per kilogram body weight, with a preferred dosage range between about 1.0 mg to about 10 mg per kilogram body weight per day (from about 0.1 g to about 1.0 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other therapeutic compounds to provide an operative combination. It is intended to include any chemically compatible combination of pharmaceutically-active agents, as long as the combination does not eliminate the activity of the compound of formula I of this invention.

The invention will now be described by way of reference only to the following general methods and experimental examples.

General Methods

Peptide Synthesis

Cyclic peptide compounds of formula I are prepared according to methods described in detail in our earlier applications No. PCT/AU98/00490 and No. PCT/AU02/01427, the entire disclosures of which are incorporated herein by this reference. While the invention is specifically illustrated with reference to the compound AcF-[OPdChaWR] (PMX53), whose corresponding linear peptide is Ac-Phe-Orn-Pro-dCha-Trp-Arg, it will be clearly understood that the invention is not limited to this compound.

Compounds 1-6, 17, 20, 28, 30, 31, 36 and 44 disclosed in International patent application No. PCT/AU98/00490 and compounds 10-12, 14, 15, 25, 33, 35, 40, 45, 48, 52, 58, 60, 66, and 68-70 disclosed for the first time in International patent application No PCT/AU02/01427 have appreciable antagonist potency (IC50<1 μM) against the C5a receptor on human neutrophils. PMX53 and compounds 33, 45 and 60 of PR8334 are most preferred.

We have found that all of the compounds of formula I which have so far been tested have broadly similar pharmacological activities, although the physicochemical properties, potency, and bioavailability of the individual compounds varies somewhat, depending on the specific substituents. In view of the structural similarities, and the demonstrated functional similarities in the context of C5a antagonist activity, it will be appreciated by those skilled in the art that the activity of PMX53 demonstrated herein is anticipated for the other compounds within the defined class of Formula I.

The general tests described below may be used for initial screening of candidate inhibitor of C5a receptors.

Drug Preparation and Formulation

The human C5a receptor antagonist AcF-[OPdChaWR] (AcPhe[Orn-Pro-D-Cyclohexylalanine-Trp-Arg]) was synthesized as described in International patent application No. PCT/AU98/00490 and No. PCT/AU02/01427, purified by reversed phase HPLC, and fully characterized by mass spectrometry and proton NMR spectroscopy. The C5a antagonist was prepared in olive oil (10 mg/mL) for oral dosing and in a 30% polyethylene glycol solution (0.6 mg/mL) for SC dosing. It was prepared in a 50% propylene glycol solution (30 mg/kg) for IP injections.

Receptor-Binding Assay

Assays are performed with fresh human PMNLS, isolated as previously described (Sanderson, S. D., Kirnarsky, L., Sherman, S. A., Vogen, S. M., Prakesh, O., Ember, J. A., Finch, A. M. and Taylor, S. M. J. Med. Chem., 1995 38 3669-3675), using a buffer of 50 mM HEPES, 1 mM CaCl₂, 5 mM MgCl₂, 0.5% bovine serum albumin, 0.1% bacitracin and 100 μM phenylmethylsulfonyl fluoride (PMSF). In assays performed at 4° C., buffer, unlabelled human recombinant C5a (Sigma) or peptide, Hunter/Bolton labelled ¹²⁵I-C5a (˜20 μM) (New England Nuclear, MA) and PMNLs (0.2×10⁶) are added sequentially to a Millipore Multiscreen assay plate (HV 0.45) having a final volume of 200 μL/well. After incubation for 60 min at 4° C., the -samples are filtered and the plate washed once with buffer. Filters are dried, punched and counted in an LKB gamma counter. Non-specific binding is assessed by the inclusion of 1 mM peptide or 100 nM C5a, which typically results in 10-15% total binding.

Data are analysed using non-linear regression and statistics with Dunnett post-test.

Receptor Agonist Activity Assay

The C5a receptor agonist activity of compounds is determined for example using the calcium rise assay disclosed in Seligmann et al. (Agents and Actions (1987). 21:375-378) or the following myeloperoxidase release assay.

Myeloperoxidase Release Assay for Antagonist Activity

Cells are isolated as previously described (Sanderson et al, 1995) and incubated with cytochalasin B (5 μg/mL, 15 min, 37° C.). Hank's Balanced Salt solution containing 0.15% gelatin and peptide is added on to a 96 well plate (total volume 100 μL/well), followed by 25 μL cells (4×10⁶/ml). To assess the capacity of each peptide to antagonise C5a, cells are incubated for 5 min at 37° C. with each peptide, followed by addition of C5a (100 n) and further incubation for 5 min. Then 50 μL of sodium phosphate (0.1 M, pH 6.8) is added to each well, the plate was cooled to room temperature, and 25 μL of a fresh mixture of equal volumes of dimethoxybenzidine (5.7 mg/ml) and H₂O₂ (0.51%) is added to each well. The reaction is stopped at 10 min by addition of 2% sodium azide. Absorbances are measured at 450 nm in a Bioscan 450 plate reader, corrected for control values (no peptide), and analysed by non-linear regression.

Statistical Analysis

Values are means± standard error mean (SEM), and differences between group means were considered significant at P<0.05. Data were analysed by a one-way ANOVA, and individual group comparisons by Student's t Test.

### EXAMPLE 1

### Inhibitory Effects of PMX53 on Secondary Lung Injury After Cutaneous Burns in Rats

Female Wistar rats of body weight 250-300 grams were used in this study. The rats were divided into three groups:

| | |
|---|---|
| Group 1 | burn-only (n = 4); |
| Group 2 | burn plus PMX53 treatment (n = 4); and |
| Group 3 | control rats without burn or drug treatment |
| | (n = 2). |

PMX53 treatment involved a subcutaneous injection of PMX53 in distilled water at a dose of 10 mg/kg 30 minutes before the burn.

With the rats under deep anaesthesia, a closely-clipped area of skin on the back equivalent to 30% of total skin area was exposed to water at a temperature of 75° C. for 30 seconds. This resulted in a full thickness skin burn (Schmid et al, Shock 8(2): 119-124, 1997). To prevent rapid death from the burn, rats were immediately treated with an intraperitoneal infusion of 8-10 ml normal saline. At the same time Evans Blue (EB) at a concentration of 20 mg/kg was injected via the femoral vein. The rats were then kept on a heating pad to maintain normal body temperature, and monitored for 4 hours. Anaesthetic was topped up as required. At the end of the experiment, 1-2 ml of blood was taken and the serum/plasma stored at −20° C., for subsequent assay of serum TNF-α levels. The leakage of EB/albumin from the blood vessels into the subcutaneous tissue was estimated by examining photographs of skin samples.

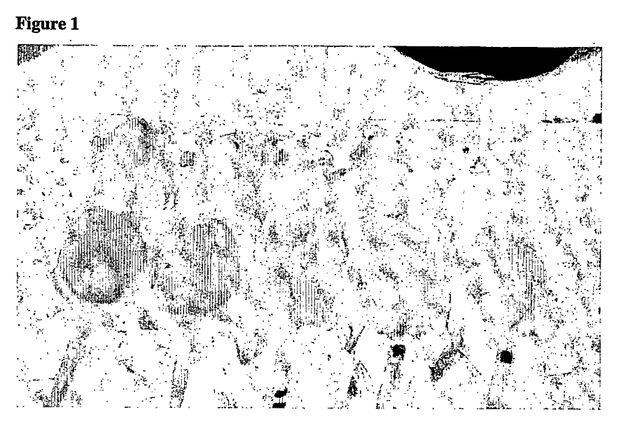

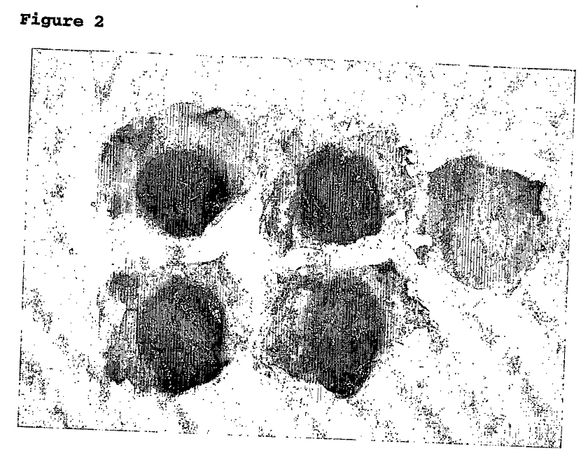

Vascular leakage into the subcutaneous tissue was indicated by blue staining, as illustrated in FIGS. 1 and 2. As shown in FIG. 1, in the burn-only group, EB distributed immediately to the entire area of the burn. After 1 hour, the blue staining was obvious and the skin became thickened and oedematous. The PMX53-treated group showed less blue staining and less thickening in the burnt skin compared to the burn-only group. At 4 hours after burning, when the animals were killed and autopsied, there was no appreciable difference in the degree of EB infiltration in the subcutaneous tissues between drug-treated and untreated burned rats, as shown in FIG. 2.

A separate series of experiments was performed in the same way to determine myeloperoxidase (MPO) levels in the lung. In this experiment the groups were as follows:

| | |
|---|---|
| Group 1 | burn (n = 6); |
| Group 2 | burn + PMX53 (n = 6); and |
| Group 3 | control (n = 6). |

Immediately after the 4 hr monitoring period following the burn the lungs were flushed with 10 ml of saline via the pulmonary artery. Bronchoalveolar lavage (BAL) fluid was collected by an irrigation of 1 ml of saline at 37° C. into the lung once through the trachea and the total number of cells present in the lavage fluid was determined. Approximately 50% of the left lung was weighed, then homogenized in 1 ml solution of 0.05% sodium azide in 0.1M PBS (pH 6.4), and then sonicated and centrifuged. The MPO levels in the supernatants of lungs were determined using a tissue MPO assay, and the results were calculated as absorbance/tissue weight (g). Samples of affected skin, lung, liver and kidney were collected for histopathology.

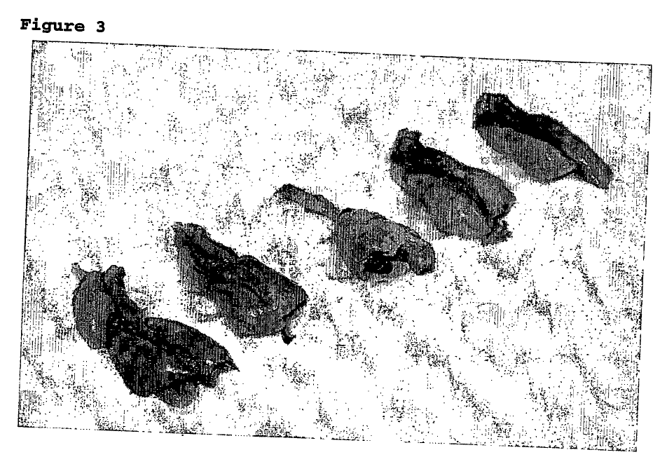

The results of the Evans Blue experiments are illustrated in FIG. 3. Rats pretreated with PMX53 had lungs of colour and texture similar to those of the normal lungs. The lungs from the burn-only group showed a greater degree of EB staining and consolidation compared to either lungs from drug-treated rats or the no-burn control rats.

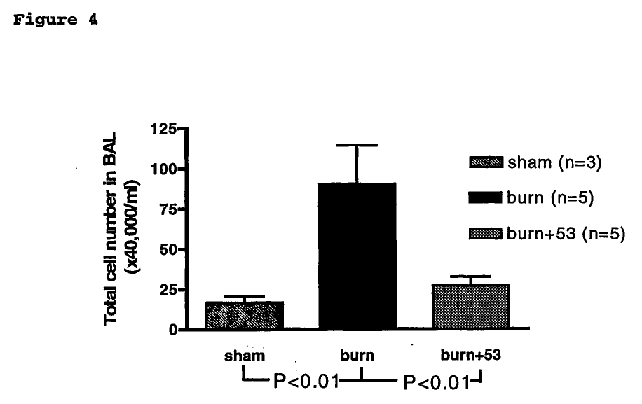

FIG. 4 illustrates the results of the cell number estimation from BAL fluid from sham operated, burned and burned and PMX53-treated rats. At 4 hours after the burn injury, the number of cells present in the BAL fluid of the PMX35-treated rats was dramatically less than the number present in untreated burned animals.

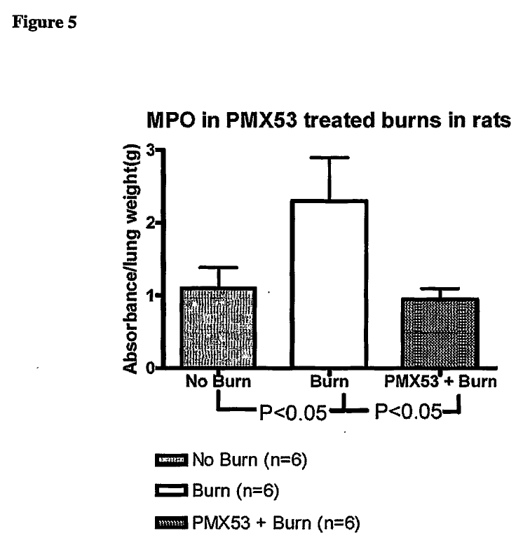

As illustrated in FIG. 5, PMX53 also significantly inhibited the increase in the MPO levels in the lungs of treated rats, compared to burn-only rats (p<0005, as assessed by ANOVA). Thus there was a protective effect of PMX53 against neutrophil infiltration.

This study also demonstrated that pre-injection of PMX53 administered subcutaneously significantly inhibited the release of MPO in the lungs 4 hours after severe burns (30% of surface area & secondary degree).

Further experiments are being conducted to expand on this finding over longer time courses following burns and in other tissues, such as liver, kidney and bowel.

Histopathological examination of skin, lung, bowel, liver and kidney samples is performed to assess the degree of inflammation and the degree of neutrophil infiltration into each tissue. AS-D Napthol staining can be used to identify PMNLs in tissue sections.

### EXAMPLE 2

### Determination of Pulmonary Permeability

For the determination of pulmonary permeability, animals are given ¹²⁵I albumin (˜1 μCi) via a tail vein catheter, and are allowed to stabilize for 30 min to establish postoperative equilibrium. During the stabilization and experimental periods, lung perfusate is collected every 10 min. Throughout the experimental period, samples of blood (0.3 ml) are withdrawn at 1 hour intervals. The blood samples are used for the measurement of total albumin concentration, and the specific activity of ¹²⁵I-albumin is used for the calculation of pulmonary albumin loss, as described below.

The heart and lungs are excised in toto, the left lung is lavaged three times with 3.5 ml Ringer's lactate solution, and the effluent bronchoalveolar lavage (BAL) fluid is collected. Blood and BAL fluid are weighed and counted for ¹²⁵I activity, and the lung permeability index (LPI) is calculated using the following formula:
LPI=BAL-¹²⁵I(cpm/g)/blood-¹²⁵I (cpm/g).

It will be appreciated that Evans Blue could alternatively be used.

### EXAMPLE 3

### Inhibitory Effects of PMX53 on Burns Through Topical Administration

The study in Example 1 demonstrated that pre-injection of PMX53 subcutaneously significantly inhibited the release of MPO in the lungs 4 hours after severe burns (30% of surface area & secondary degree). However, the neutrophil infiltration in the burned area was not apparent in this model. It was also of interest to determine whether systemic administration of a C5a antagonist was required for the treatment or prevention of organ dysfunction in burned patients, since it may be advantageous for patients not to have systemic suppression of aspects of their immune system after a severe burn.

The inhibitory effect of topically applied PMX53 on neutrophil infiltration following a burn was examined after a 6 hour period. In order to verify any effect of PMX53 on the immune system, immunoglobulin 4 (IgG) levels were examined. IgM levels can be examined using similar methods.

The kinetics of the passage of PMX53 into the bloodstream following topical administration on burned rat skin were also examined. Previous experiments had demonstrated that in the rat topical administration of PMX53 results in a lower systemic level of the drug compared with administration by other routes.

Female Wistar rats of body weight about 250 grams were used in this study. A total of nine rats were used in the experiments, divided into 3 groups of 3 animals each:

| | |
|---|---|
| Group 1 | no burn; |
| Group 2 | burn only; and |
| Group 3 | burn plus PMX53 treatment; |

Both sides of the anaesthetized rat body was shaved. Three spots along the middle part of each side of the rat body were then burned using heated brass weights (treated using 100° C. water) 1 cm in diameter, 2 cm in height, 30 grams in weight for 10 seconds. This resulted in second degree burns over 15% of the surface area of the rat. For the drug-treated group, 40 μl of PMX53 solution (400 μg/spot, 10 mg PMX53/ml in a solution containing 30% propylene glycol in distilled water) was applied on the burned skin immediately following the burns.

Rats were then kept on a heating pad and closely monitored for 6 hours. At the end of the experiments, plasma or serum was taken for immunoglobulin measurement and for analysis of the levels of circulating PMX53. Skin samples were collected for histopathology.

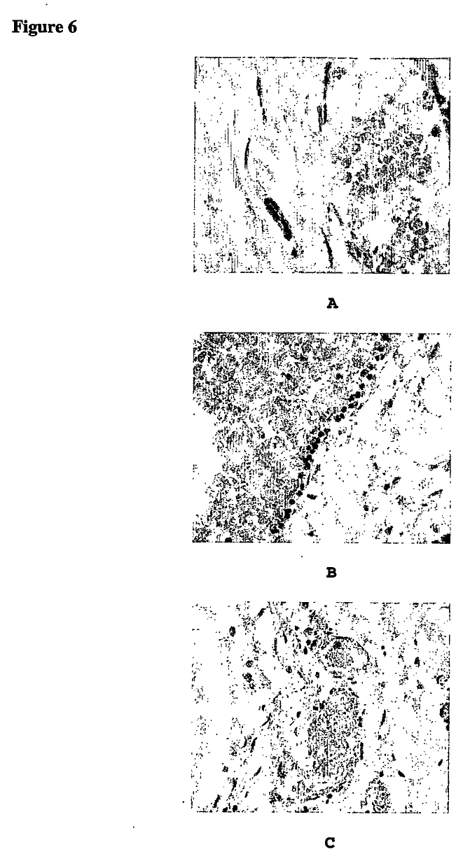

The results of these experiments are illustrated in FIG. 6.

Unburned skin showed normal structures on histological examination. Only a few neutrophils were seen, and these were mainly inside the vessels (FIG. 6A).

Burned skin showed a disorganized structure and edema. Margination of neutrophils was seen in the vessels in the deep muscle layer with some neutrophils scattered around (FIG. 6B).

PMX53-treated skin showed the same structural damage as burned skin, but there appeared to be less neutrophil recruitment (FIG. 6C). Few neutrophils had migrated to the burned tissue 6 hours after the burn; however, this may be due to the relatively short period.

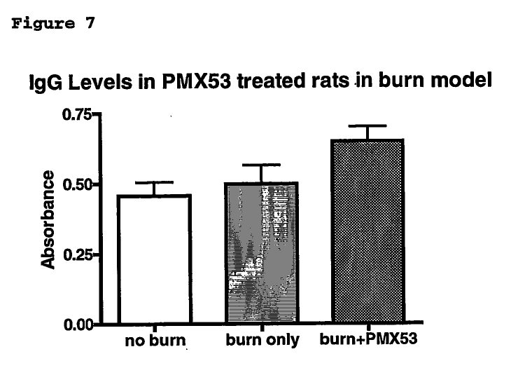

The change in circulating IgG levels following the administration of PMX53 are illustrated in FIG. 7. IgG levels in plasma were measured using an ELISA assay. IgM levels may also may be determined using conventional ELISA techniques. There was no suppression of IgG over the 6 hr period after burns to 15% of the rat skin surface area (n=3). Topical administration of PMX53 at a dose of 400 μg/site (total of 6 sites, 2.4 mg/rat) did not cause any decline of the IgG levels in the same rat model. The results here suggest that the full thickness skin burn to less than 15% has little effect on systemic immunoglobulin levels in the rat model. The 6 hr period may be too short for the systemic reaction to the burns to be detectable.

Similar experiments may be carried out over longer periods, such as 12, 18, 24, 36 and 48 hours, to determine whether there are changes in levels of circulating IgG and IgM over longer periods of time. ELISA assay kits for quantifying levels of human IgG and IgM are readily available (see for example Bethyl Laboratories, Human IgM ELISA Quantitation Kit and Human IgG ELISA Quantitation Kit).

The following table summarizes the findings of the determination of PMX53 entering the bloodstream following topical administration to burned skin.

TABLE 1

| | PMX53 in | | PMX53 in | |
|---|---|---|---|---|
| | blood | Penetration | blood | Penetration |
| | (μg/ml) | (%/cm²) | (μg/ml) | (%/cm²) |
| | t = 30 min | t = 30 min | t = 60 min | t = 60 min |
| Rat 1 | 0.0286 | 3.7 | 0.00834 | 1.11 |
| Rat 2 | 0 | 0 | 0.0878 | 11.43 |
| Rat 3 | 0.00927 | 1.23 | 0.00688 | 0.92 |

The penetration value for each time point was calculated using the concentration of PMX53 in the blood (A), assuming that the blood volume took up 6% of the 250 g body weight (B) and factoring in the dose applied (C) and the total surface area of skin covered by the dose (D), using the formula:

$$\frac{A \times 15 \times 100 \%}{C \times D}$$

The degree of penetration of PMX53 through the burned rat skin showed a large variation, which indicated that topical application of the drug on burned patients may increase the systemic level of the drug. The penetration of the compound through burned rat skin was significantly higher than the penetration through normal rat skin (0.16%/cm², at 60min). Accordingly, the dose of the topically applied compound and the size of the surface area for administration on burned patients will have to be carefully adjusted for safety. These results may not reflect the same result as with human skin because of differences between the responses of rat skin and human skin. For instance, second degree burned rat skin does not blister; however, a person of skill in the art would readily be able to determine dosages for topical administration using only routine methods.

The relatively rapid penetration of PMX53 into the bloodstream following topical administration may prove advantageous, as it demonstrates that this molecule is readily able to be distributed systemically, unlike larger molecules such as immunoglobulins.

### EXAMPLE 4

### Interaction Between PMX53 and Silver Coated Wound Dressing

Acticoat(™, Smith and Nephew) antimicrobial dressings provide sustained protection of a wound site from external bacterial contamination. The antimicrobial barrier remains effective for up to 7 days.

As silver ions are leached from the surface of the Acticoat(™) wound dressing, it was of interest to determine whether PMX53 had any effect on this process or otherwise interacted with the silver coating that may lead to a reduction in wound healing or antibacterial properties.

Uniformly sized segments of the Acticoat dressing (10 mm diameter) were incubated in 7 ml PMX53 solution (1.0 mM in sterile water) or sterile water alone at 37° C. for periods of up to 7 days. The segments were then removed and examined using scanning electron microscopy to determine if the silver coating was affected.

A quantitative assessment of the effect of PMX53 on silver ion leaching was performed by determining the concentration of silver ions in each solution after the incubation described above for 1, 3, 5 and 7 days. Silver ion determination was performed using a Spectroflame model P ICPAES instrument.

An analysis of the concentration of PMX53 remaining in the incubation solutions was performed by high performance liquid chromatography to ascertain if the concentration of PMX53 remained constant throughout the incubation period. PMX53 solution was incubated at 37° C. without a dressing to act as a control.

There were no apparent changes at the surface of Acticoat(TM) dressing segments after 7 days incubation with PMX53 visible by scanning electron microscopy at a magnification of 2000.

The results of the silver leaching experiments are provided in Table 2. The presence of PMX53 (1 mM) in the incubation solution increased the leeching of silver ions from the Acticoat(TM) dressing pieces by a factor of 2.5 after 1 day of incubation at 37° C. Thereafter the concentration of silver ions in the incubating solutions did not change to any extent from Day 1 until Day 7.

TABLE 2

| Sample | Time | Silver ion content (mg/L) |
|---|---|---|
| Acticoat | Day 1 | 19.8 |
| Acticoat + PMX53 | Day 1 | 49.7 |
| Acticoat | Day 3 | 16.2 |
| Acticoat + PMX53 | Day 3 | 46.5 |
| Acticoat | Day 5 | 14.2 |
| Acticoat + PMX53 | Day 5 | 48.5 |
| Acticoat | Day 7 | 14.0 |
| Acticoat + PMX53 | Day 7 | 46.6 |

The results of the assessment of the effect of Acticoat(™) dressing on the concentration of PMX53 in the incubation solution are presented in Table 3. The concentration of PMX53 incubated with the Acticoat(™) dressing did not change throughout the experiment when compared to the control PMX53 solution. It is likely that the PMX53 is not degraded by incubation with Acticoat(™) dressing segments.

TABLE 3

| | | PMX53 Concentration |
|---|---|---|
| Sample | Time | (% of control) |
| Acticoat + PMX53 | Day 1 | 98.0 |
| Acticoat + PMX53 | Day 5 | 97.5 |
| Acticoat + PMX53 | Day 7 | 110.9 |

In these studies we have demonstrated for the first time that a systemic injury, such as organ dysfunction, which arises from severe burns can be attenuated using a specific small molecule C5a receptor antagonist, PMX53.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

1. A method of treatment of a systemic injury secondary to burns, comprising the step of administering to a subject in need thereof an effective amount of a compound which is an antagonist of a C5a receptor and which is a cyclic peptide or peptidomimetic compound of Formula I: 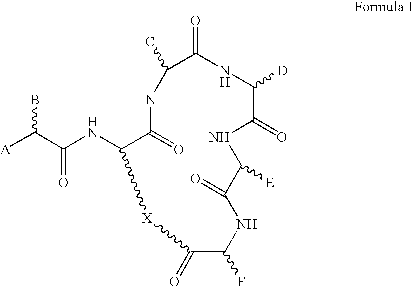
where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;
B is an alkyl, aryl, phenyl, benzyl, naphthyl or indole group, or the side chain of a D- or L-amino acid, but is not the side chain of glycine, D-phenylalanine, L-homophenylalanine, L-tryptophan, L-homotryptophan, L-tyrosine, or L-homotyrosine;
C is the side chain of a D-, L- or homo-amino acid, but is not the side chain of isoleucine, phenylalanine, or cyclohexylalanine;
D is the side chain of a neutral D-amino acid, but is not the side chain of glycine or D-alanine, a bulky planar side chain, or a bulky charged side chain;
E is a bulky substituent, but is not the side chain of D-tryptophan, L-N-methyltryptophan, L-homophenylalanine, L-2-naphthyl L-etrahydroisoquinoline, L-cyclohexylalanine, D-leucine, L-fluorenylalanine, or L-histidine;
F is the side chain of L-arginine, L-homoarginine, L-citrulline, or L-canavanine, or a bioisostere thereof; and
X is —(CH₂)ₙNH— or (CH₂)ₙ—S—, where n is an integer of from 1 to 4; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

2. A method according to claim 1, in which n is 2 or 3.

3. A method according to claim 1, in which A is an acetamide group, an aminomethyl group, or a substituted or unsubstituted sulphonamide group.

4. A method according to claim 1, in which A is a substituted sulphonamide, and the substituent is an alkyl chain of I to 6 carbon atoms, or a phenyl or toluyl group.

5. A method according to claim 4, in which the substituent is an alkyl chain of 1 to 4 carbon atoms.

6. A method according to claims 1, in which B is the side chain of L-phenylalanine or L-phenylglycine.

7. A method according to claims 1, in which C is the side chain of glycine, alanine, leucine, valine, proline, hydroxyproline, or thioproline.

8. A method according to claims 1, in which D is the side chain of D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine.

9. A method according to claims 1, in which E is the side chain of an amino acid selected from the group consisting of L-phenylalanine, L-tryptophan and L-homotryptophan, or is L-1-napthyl or L-3-benzothienyl alanine.

10. A method according to of claims 1, in which the compound has no detectable agonist activity at the C5a receptor.

11. A method according to claims 1, in which the compound has a receptor affinity IC₅₀<25 μM, and an antagonist potency IC_{50.}<1 μ\tilde{M}

12. A method according to claims 1, in which the compound is selected from the group consisting of compounds 1 to 6, 10 to 15, 17, 19, 20, 22, 25, 26, 28, 30, 31, 33 to 37, 39 to 45, 47 to 50, 52 to 58 and 60 to 70 described in PCT/AU02/01427.

13. A method according to claim 12, in which the compound is AcF[OP-DCha-WR], AcF[OP-DPhe-WR], AcF[OP-DCha-FR], AcF[OP-DCha-WCit]), HC-[OpdChaWR], AcF-[OpdPheWR], AcF-[OpdChaWCitrulline] or HC-[OpdPheWR].

14. A method according to claims 1, in which the systemic injury is organ dysfunction or failure.

15. A method according to claims 1, in which the treatment is a prophylactic treatment.

16. A method according to claims 1, in which the treatment is a therapeutic treatment.

17. A method according to claims 1, in which the organ dysfunction or failure is selected from the group consisting of any one or more of lung, kidney, liver and bowel dysfunction or failure.

18. A method according to claim 17, in which the organ dysfunction or failure is lung dysfunction or failure.

19. A method according to claims 1, in which the subject is a human.

20. A method according to claims 1, in which the inhibitor is administered intravenously, orally, subcutaneously, transdermally, or topically.

21. A method according to claims 1, in which the inhibitor is administered intravenously or topically.

22. (canceled)

23. (canceled)

24. (canceled)

25. (canceled)

26. A pharmaceutical or veterinary agent for treating a systemic injury secondary to burns, comprising a compound which is an antagonist of a C5a receptor and which is a cyclic peptide or peptidomimetic compound of Formula l: 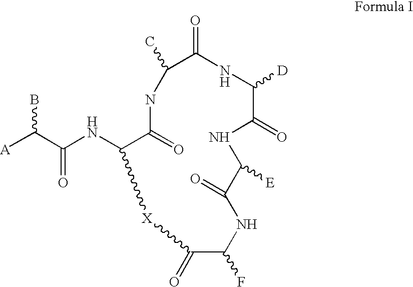
where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;
B is an alkyl, aryl, phenyl, benzyl, naphthyl or indole group, or the side chain of a D- or L-amino acid, but is not the side chain of glycine, D-phenylalanine, L-homophenylalanine, L-tryptophan, L-homotryptophan, L-tyrosine, or L-homotyrosine;
C is the side chain of a D-, L- or homo-amino acid, but is not the side chain of isoleucine, phenylalanine, or cyclohexylalanine;
D is the side chain of a neutral D-amino acid, but is not the side chain of glycine or D-alanine, a bulky planar side chain, or a bulky charged side chain;
E is a bulky substituent, but is not the side chain of D-tryptophan, L-N-methyltryptophan, L-homophenylalanine, L-2-naphthyl L-etrahydroisoquinoline, L-cyclohexylalanine, D-leucine, L-fluorenylalanine, or L-histidine;
F is the side chain of L-arginine, L-homoarginine, L-citrulline, or L-canavanine, or a bioisostere thereof; and
X is —(CH₂)ₙNH— or (CH₂)ₙ—S—, where n is an integer of from 1 to 4; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

27. A composition for treating a systemic injury secondary to burns, comprising a compound which is an antagonist of a C5a receptor and which is a cyclic peptide or peptidomimetic compound of Formula I: 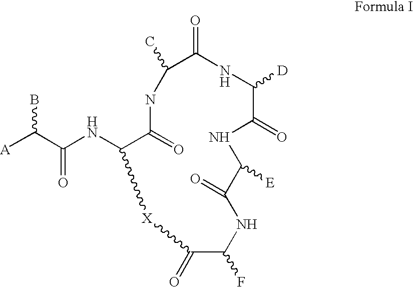
where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;
B is an alkyl, aryl, phenyl, benzyl, naphthyl or indole group, or the side chain of a D- or L-amino acid, but is not the side chain of glycine, D-phenylalanine, L-homophenylalanine, L-tryptophan, L-homotryptophan, L-tyrosine, or L-homotyrosine;
C is the side chain of a D-, L- or homo-amino acid, but is not the side chain of isoleucine, phenylalanine, or cyclohexylalanine;
D is the side chain of a neutral D-amino acid, but is not the side chain of glycine or D-alanine, a bulky planar side chain, or a bulky charged side chain;
E is a bulky substituent, but is not the side chain of D-tryptophan, L-N-methyltryptophan, L-homophenylalanine, L-2-naphthyl L-etrahydroisoquinoline, L-cyclohexylalanine, D-leucine, L-fluorenylalanine, or L-histidine;
F is the side chain of L-arginine, L-homoarginine, L-citrulline, or L-canavanine, or a bioisostere thereof; and
X is —(CH₂)ₙNH— or (CH₂)ₙ—S—, where n is an integer of from 1 to 4; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid,
together with a pharmaceutically or veterinarily-acceptable carrier.

28. A composition according to claim 27, which is formulated for topical administration.

29. A composition according to claim 27, which is in the form of a bandage or dressing.
